(19) United States
(12) Patent Application Publication
(10) Pub. No.: US 2006/0246115 A1
(43) Pub. Date: Nov. 2, 2006

(54) Nutritional products for ameliorating symptoms of rheumatoid arthritis

(76) Inventors: Ricardo Rueda, Granada (ES); Alejandro Barranco, Las Gabias (ES); Maria Ramirez, Granada (ES)

Correspondence Address: ROSS PRODUCTS DIVISION OF ABBOTT LABORATORIES;DEPARTMENT 108140-DS/1, 625 CLEVELAND AVENUE, COLUMBUS, OH, 43215-1724, US

(21) Appl. No.: 11/367,982

(22) Filed: Mar. 3, 2006

Related U.S. Application Data
(60) Provisional application No. 60/658,931, filed on Mar. 4, 2005.

(51) Int. Cl. A61K 36/324; A61K 36/32; A61K 31/202

(52) U.S. Cl. 424/439; 424/725; 514/560

(57) ABSTRACT

Disclosed are nutritional compositions for treating rheumatic diseases. The nutritional compositions contain a fat source containing at least one of: 1) at least one omega-3 long chain polyunsaturated fatty acid and 2) at least one omega-6 long chain polyunsaturated fatty acid; a carbohydrate source; a protein source; and at least one of a Boswellia extract and a Phlebodium extract. Also disclosed are methods of treating rheumatic diseases and symptoms thereof, involving administering to a subject an effective amount of the aforementioned nutritional composition.

This application claims the benefit of U.S. Provisional Application No. 60/658,931 filed Mar. 4, 2005

### FIELD OF THE INVENTION

The present invention relates to nutritional compositions and corresponding methods of using those compositions to ameliorate symptoms of arthritis or other rheumatic diseases or conditions in afflicted by or at risk of developing such diseases or conditions.

### BACKGROUND OF THE INVENTION

Connective tissue is a reference to the tissues that hold a body together. Connective tissue disease is the term used to generally describe a long list of afflictions that involve connective tissue. Millions of people suffer pain due to inflammation of connective tissue, particularly pain in the joints. The pain ranges from mild soreness to debilitating pain that can prevent any motion of the afflicted body part.

Rheumatic diseases, which are one type of connective tissue disease, include a variety of different conditions. A common feature of rheumatic diseases is the involvement of joints and the surrounding tissues such as ligaments, tendons and muscles. Rheumatic diseases are usually divided into those that primarily involve joints, known as arthritis, and those involving other tissues, generally referred to connective tissue diseases. Arthritis is further subdivided into inflammatory and non-inflammatory arthritis.

Osteoarthritis is a non-inflammatory type of arthritis. Osteoarthritis is generally considered to be due to degradation by extended use of the joints leading to damage of the joint surfaces, which results in pain on movement of the joint. Symptoms in osteoarthritis tend to get worse with activity, so that the greatest pain is experienced at the end of the day. In contrast, the symptoms of inflammatory arthritis include the greatest pain occurring at the movement of a joint after a night's sleep of inactivity.

Inflammatory arthritis generally means those diseases of joints where, for example, the immune system and/or some other mechanism(s) are causing inflammation in the joint. Among the more common types of inflammatory arthritis are rheumatoid arthritis, gout, psoriatic arthritis (associated with the skin condition psoriasis), reactive arthritis, viral or post-viral arthritis (occurring after an infection), and spondylarthritis, which affect the spine as well as joints.

The characteristic symptoms of inflammatory arthritis are pain and swelling of one or more joints. The afflicted joints are often warmer than the other joints of the body. Alternatively or additionally, stiffness of the afflicted joints often occurs upon waking in the morning, or after remaining stationary for a period of time. At this time, there are no certain and identifiable cause associated with the commencement of inflammatory arthritis.

While not often referred to as formal diseases, there are a number of minor pains that are not classified as arthritis but are due to injury, strain, and/or inflammation (of connective tissue) but nevertheless are referred to as soft tissue rheumatism, some examples of which include tennis elbow, frozen shoulder, carpal tunnel syndrome, plantar fasciitis, and Achilles tendonitis.

### SUMMARY OF THE INVENTION

The present invention is directed to nutritional compositions, including medical foods, comprising a fat source, a carbohydrate source, and protein source, wherein the compositions include at least one omega-3 long chain polyunsaturated fatty acid, optionally an omega-6 long chain polyunsaturated fatty acid such as gamma-linolenic acid (GLA), and at least one of a Boswellia extract and a Phlebodium extract.

The present invention is also directed to methods of ameliorating the symptoms of arthritis or other rheumatic diseases or conditions, by administration to such individuals afflicted by or at risk of developing such diseases or conditions, a nutritional composition comprising a fat source, a carbohydrate source, and protein source, wherein the composition includes at least one omega-3 long chain polyunsaturated fatty acid, optionally an omega-6 long chain polyunsaturated fatty acid such as gamma-linolenic acid (GLA), and at least one of a Boswellia extract and a Phlebodium extract.

The nutritional compositions and corresponding methods of the present invention are useful in ameliorating the symptoms of arthritis or other rheumatic diseases or conditions in individuals afflicted by or at risk of developing such diseases or conditions. Without being limited by theory, it is believed that these compositions and corresponding methods provide ant-inflammatory activity and specifically decrease the content of pro-inflammatory cytokines in joints.

### BRIEF DESCRIPTION OF THE DRAWINGS

FIG. 1 graphically shows, from Experiment 1, the incidence and day of arthritis onset in type II collagen-immunized mice fed with control diet, the same diet supplemented with Boswellia, Curcuma, Crataeva or Polyphenol extracts, and immunized mice with Oxepa®. The Prednisolone group is immunized mice fed with control diet and treated intraperitoneally with prednisolone daily (* denotes statistical significance relative to control at p<0.05).

FIG. 2 graphically shows, from Experiment 1, the arthritis index and relative severity in type II collagen-immunized mice fed with control diet, the same diet supplemented with Boswellia, Curcuma, Crataeva or Polyphenol extracts, and immunized mice fed with Oxepa®. The Prednisolone group is immunized mice fed with the control diet and treated intraperitoneally with prednisolone daily (* denotes statistical significance relative to control at p<0.05).

FIG. 3 graphically shows, from Experiment 1, anti-CII IgG2a concentrations in serum and joint homogenates of type II collagen-immunized mice fed with control diet, the same diet supplemented with Boswellia, Curcuma, Craeva or Polyphenol extracts, and immunized mice fed with Oxepa®. The Prednisolone group is immunized mice fed with control diet and treated intraperitoneally with prednisolone daily (* denotes statistical significance relative to control at p<0.05).

FIG. 4 graphically shows, from Experiment 1, the concentration of IL-1β, IL-6, IL-10 and MMP-9 in joint homogenates of type II collagen immunized mice fed with control diet, the same diet supplemented with Boswellia, Curcuma, Crataeva or Polyphenol extracts, and immunized mice fed with Oxepa®. The Prednisolone group is immunized mice fed with control diet and treated intraperitoneally with prednisolone daily (* denotes statistical significance relative to control at p<0.05).

FIG. 5 graphically shows, from Experiment 2, the incidence and day of arthritis onset in type II collagen-immunized mice fed with control diet or the same diet supplement with Phlebodium extract. (* denotes statistical significance relative to control at p<0.05).

FIG. 6 graphically shows, from Experiment 2, arthritis index and relative severity in type II collagen-immunized mice fed with control diet or the same diet supplemented with Phlebodium extract.

FIG. 7 graphically shows, from Experiment 3 hereof, synovial inflammation, pannus formation, cartilage damage, bone destruction and index of morphological inflammation in type II collagen-immunized mice fed with control diet, the same diet supplemented with Boswellia, Polyphenol or Phlebodium decumanum extracts, and immunized mice fed with Oxepa®. The Prednisolone group (Pred) is immunized mice fed with the control diet and treated intraperitoneally with prednisolone daily. Data are mean±SEM (*denotes statistical significance relative to control at p<0.05).

FIG. 8 shows a histological slide from Experiment 3 (Control group) of a knee from the right hind limb of a subject mouse showing severe inflammation (degree 3), mild pannus formation (degree 1), severe cartilage damage (degree 3), and very mild bone destruction (degree 1). H&E. 4×.

FIG. 9 shows a histological slide from Experiment 3 (Corticoid group) of a knee from the left hind limb of a subject mouse showing a normal joint. H&E. 4×.

FIG. 10 shows a histological slide from Experiment 3 (Boswellia group) of a tarsal joint from the left hind limb of a subject mouse showing mild inflammation (degree 1), mild cartilage damage (degree 1), and normal bone structure. H&E. 4×.

FIG. 11 shows a histological slide from Experiment 3 (Polyphenol group) of a knee from the left hind limb of a subject mouse showing severe inflammation (degree 3), moderate pannus formation (degree 2), severe cartilage damage (degree 3), and no bone affectation. H&E. 4×.

FIG. 12 shows a histological slide from Experiment 3 (Phlebodium group) of an elbow from the right fore limb of a subject mouse showing severe inflammation (degree 3), moderate cartilage damage (degree 2), and mild bone affectation (degree 1). H&E. 4×.

FIG. 13 shows a histological slide from Experiment 3 (Oxepa group) of an elbow from the right fore limb of a subject mouse showing mild synovial inflammation with few inflammatory cells (degree 1). H&E. 10×.

### DETAILED DESCRIPTION OF THE INVENTION

The compositions and corresponding methods of the present invention are directed to nutritional compositions that contain as essential elements a fat source, a protein source, and a carbohydrate source, including an omega-3 long chain polyunsaturated fatty acid and an omega-6 long chain polyunsaturated fatty acid, and at least one of a Boswellia extract and a Phlebodium extract. These and other essential or optional elements or limitations of the compositions and corresponding methods of the present invention are described in detail hereinafter.

The terms “ameliorating ” or “ameliorate ” as use herein, unless otherwise specified, mean treating, controlling, preventing, or otherwise reducing the occurrence, severity or relapse of an identified symptom, condition, or disease, in individuals afflicted with or prone to develop such symptoms, condition or disease.

The term “medical food: ” as used herein, unless otherwise specified, refers generally to food that is formulated to be consumed or administered enterally under the supervision of a physician and that is intended for the specific dietary management of a disease or condition for which distinctive nutritional requirements, based on recognized scientific principles, are established by medical evaluation.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Any reference to singular characteristics or limitations of the present invention shall include the corresponding plural characteristic or limitation, and vice versa, unless otherwise specified or clearly implied to the contrary by the context in which the reference is made.

Any combination of method or process steps as used herein may be performed in any order, unless otherwise specified or clearly implied to the contrary by the context in which the referenced combination is made.

The compositions and methods of the present invention may comprise, consist of, or consist essentially of the essential elements and limitations of the invention described herein, as well as any additional or optional ingredients, components, or limitations described herein or otherwise useful in a nutritional or pharmaceutical application.

The compositions and methods of the present invention may also be substantially free of any optional ingredients described herein. In this context, the term “substantially free ” means that the selected composition contains less than a functional amount of the optional ingredient preferably zero percent by weight of such optional ingredient.

### Product Form

The nutritional compositions of the present invention are directed to any known or otherwise suitable product form for oral administration. Any solid, liquid, or powder form, including combinations or variations thereof, are suitable for use herein, provided that such forms allow for safe and effective oral delivery of the essential and other selected ingredients in the targeted product form.

Non-limiting examples of solid nutritional product forms suitable for use herein include snack and meal replacement products, including those formulated as bars, sticks, cookies or breads or cakes or other baked goods, frozen liquids, candy, breakfast cereals, powders or granulated solids or other particulates, snack chips or bites, and so forth. The nutritional compositions may also be formulated into other product forms such as capsules, tablets, caplets, and so forth.

Non-limiting examples of liquid nutritional product forms suitable for use herein include snack and meal replacement products such as those formulated as juices or other acidified beverages, milk or soy-based beverages, shakes, coffees, teas, carbonated beverages, non-carbonated beverages, enteral feeding compositions, and so forth. These liquid compositions are most typically formulated as suspensions or emulsions, but can also be formulated in any other suitable form such as solutions, liquid gels, and so forth.

Other non-limiting examples of suitable product forms for use herein include semi-solid or semi-liquid compositions such as puddings, gels, and so forth.

### Boswellia and Phlebodium Extracts

The nutritional compositions of the present invention comprise a Boswellia extract, a Phlebodium extract, or a combination thereof, in an amount effective to ameliorate the symptoms of arthritis or other rheumatic disease or condition. The concentration of such extracts in the compositions may range from about 0.1% to about 5%, including from about 0.2% to about 3%, and also including from about 0.3% to about 2%, by weight of the composition.

The term “extract ” as used herein, unless otherwise specified, means a concentrate of water-soluble and/or alcohol-soluble and/or other suitable solvent-soluble plant components from the portion of a plant extracted. The extract can be in liquid, paste, oil, or powdered form.

The Boswellia extract can be obtained from plants belonging to at least one of following genera: Boswellia, Commiphora, and Bursera or closely related woody plant species of the family Burseraceae. The Boswellia extract generally contains one or more boswellic acids.

The Boswellia extract is preferably obtained from the exudate gums, gum resins, or standardized gum extracts obtained or derived from a woody plant species of the family Burseraceae. The leaves, roots, and/or stems may also be obtained to obtain the extract. See, for example, Sen et al, Carbohydrate Res. 223, 321 (1992) and Ammon et al, Planta Med. 57, 203 (1991), which is hereby incorporated by reference in this regard.

The trees of the genera Boswellia, Commiphora, Bursera, or closely related woody plant species of the family Burseraceae typically grow wild in the arid and semi-arid tropics and warm temperate zones of the world and contain high concentrations of boswellic acids and other closely related compounds. Examples of specific plants botanical sources for providing the Boswellia extract include Boswellia serrata, Boswellia bhau-dajiana, Boswellia frereana, Boswellia papyrifera, Sudanese Boswellia sacra, Boswellia carteri, Commiphora incisa, Commiphora myrrha, Commiphora abyssinica, Commiphora erthraea, Commiphora molmol, and Bursera microphylla.

The nutritional compositions of the present invention include those embodiment comprising from about 10 mg to about 1800 mg, preferably from about 100 mg to about 800 mg, of Boswellia gum extract, per dose, to thus provide from the extract an effective amount of boswellic acid.

In one embodiment of the present invention, the Boswellia extract contains at least one boswellic acid having the chemical structure represented by the following Formula I: 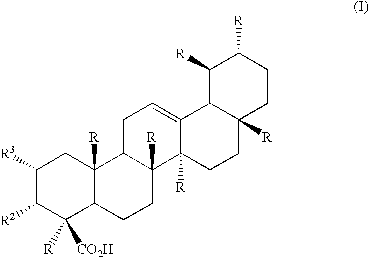
wherein each R is independently alkyl, alkenyl, aryl, alkoxy, or hydroxyalkyl; R² is hydroxy, alkoxy, hydroxyalkyl, or alkoxycarbonyl; and R³ is hydrogen, hydroxy, alkyl, alkenyl, alkoxy, or hydroxyalkyl (in each instance each of the alkyl, alkenyl, aryl, alkoxy, hydroxyalkyl, and alkoxycarbonyl groups independently contains from 1 to about 10 carbon atoms).

In another embodiment, the Boswellia extract contains at least one boswellic acid having the chemical structure represented by the following Formula II: 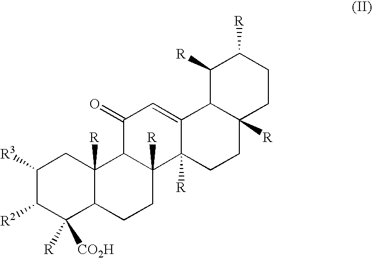
wherein each R is independently alkyl, alkenyl, aryl, alkoxy, or hydroxyalkyl; R² is hydroxy, alkoxy, hydroxyalkyl, or alkoxycarbonyl; and R³ is hydrogen, hydroxy, alkyl, alkenyl, alkoxy, or hydroxyalkyl (in each instance each of the alkyl, alkenyl, aryl, alkoxy, hydroxyalkyl, and alkoxycarbonyl groups independently contains from 1 to about 10 carbon atoms).

In another embodiment, the Boswellia extract contains at least one boswellic acid having the chemical structure represented by the following Formula III: 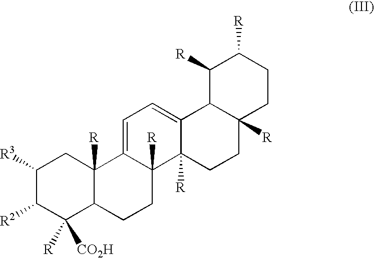
wherein each R is independently alkyl, alkenyl, aryl, alkoxy, or hydroxyalkyl; R² is hydroxy, alkoxy, hydroxyalkyl, or alkoxycarbonyl; and R³ is hydrogen, hydroxy, alkyl, alkenyl, alkoxy, or hydroxyalkyl (in each instance each of the alkyl, alkenyl, aryl, alkoxy, hydroxyalkyl, and alkoxycarbonyl groups independently contains from 1 to about 10 carbon atoms).

In yet another embodiment of the present invention, in the chemical structure represented by Formulae I, II, or III, each R is independently alkyl containing 1 to about 4 carbon atoms; R² is hydroxy, alkoxy containing 1 to about 4 carbon atoms, hydroxyalkyl containing 1 to about 4 carbon atoms, or alkoxycarbonyl containing 1 to about 4 carbon atoms; and R³ is hydrogen or hydroxy. The boswellic acids may be in free acid form, in acid salt form, or in ester form. Common examples of alkyl, alkenyl, aryl, alkoxy, hydroxyalkyl, and alkoxycarbonyl groups include methyl, ethyl, propyl, butyl, cyclohexyl, propenyl, phenyl, methoxy, ethoxy, hydroxymethyl, hydroxyethyl, and acetyl.

Boswellic acids include triterpenoic acids. Specific examples of boswellic acids include B-Boswellic acid (3α-hydroxy urs-12-ene-24-oic acid) (Formula I wherein each R is methyl, R² is hydroxy, and R³ is hydrogen); acetyl B-boswellic acid (3α-acetoxy urs-12-ene-24-oic acid) (Formula I wherein each R is methyl, R² is acetyl, and R³ is hydrogen); 11-keto-B-boswellic acid (3α-hydroxy urs-12-ene-11-keto-24-oic acid) (Formula II wherein each R is methyl, R² is hydroxy, and R³ is hydrogen); acetyl 11-keto-B-boswellic acid (3α-acetoxy urs-12-ene-11-keto-B-boswellic acid) (Formula I wherein each R is methyl, R² is acetyl, and R³ is hydrogen), 3α-hydroxy urs-9,12-diene-24-oic acid (Formula III wherein each R is methyl, R² is hydroxy, and R³ is hydrogen), and 2α, 3α dihydroxy urs-12-ene-24-oic acid (Formula I wherein each R is methyl, R² is hydroxy, and R³ is hydroxy).

The Boswellia extract may additionally or alternatively contain one or more isomers of boswellic acid or its derivatives. Examples of isomers include alpha, beta, and 11-keto-beta boswellic acid. Derivatives include acid salts, acid esters, and the acetyl and other ester derivatives.

The Boswellia extract contains at least about 10%, including from about 25% to 100%, and also including from about 40% to 80%, by weight of one or more boswellic acids, one or more boswellic acid isomers, and/or its one or more boswellic acid derivatives.

The Phlebodium extract contains a plant extract obtained from a plant within the Family Polypodiaceae. The Polypodiaceae family generally includes fems, especially those native to the tropical regions of the world. For example, many of the Polypodiaceae family are indigenous to Latin America, especially those in the Honduran rainforests, to South America especially those in the Brazilian rainforests, Mexico, and to the Caribbean islands. The Phlebodium extract is typically obtained from the rhizome or root system, and/or the leaves. The Phlebodium extract is a mixture of one or more of various flavonoids, alkaloids, and/or lipids.

Within the Family Polypodiaceae, Phlebodium extracts can be obtained from plants within the Genus Polypodium, the Genus Chrysopteris, the Subgenus Phlebodium, and other closely related fern-like plants. Specific examples Phlebodium extract include extracts from Polypodium decumanum, Phlebodium decumanum, Polypodium multiseriale, Phlebodium multiseriale, Chrysopteris decumana, Polypodium leucotomos, Phlebodium leucotomos, Polypodium aureum, Phlebodium aureum, Polypodium vulgare, Polypodium triseriale, Pteridium aquilinum, Cyathea taiwamiana, Polypodium crassifolium, Polypodium lanceolatum, Polypodium percussum, and the like.

The Boswellia and/or Phlebodium extracts can be obtained using conventional or otherwise known extraction techniques, non-limiting examples of which are described in U.S. Pat. Nos. 6,264,995; 5,932,101; 5,908,628; 5,891,440; 5,874,084 and 5,120,558, which descriptions are incorporated herein by reference. Boswellia extracts are commercially available under the tradename Boswelya Plus from Ayush Herbs, Inc., Bellevue, Wash., USA. Phlebodium extracts are commercially available under the tradename EXPLY-37® from HELSINT S.A.L., Spain.

The Boswellia and/or Phlebodium extracts may be prepared, for example, by individually washing, drying and grinding the plant material into fine powder, and then, if desired, extracting the ground plant material. An exemplary preparation of the Boswellia extract includes: crushing lumps of a resin from a plant or crushing a portion of the plant and extracting with a polar solvent; removing the insoluble material by known methods; concentrating the extracts under reduced pressure by removing the organic solvent to obtain a syrupy mass; basifying the syrupy mass with an aqueous solution of an alkali to attain a pH above 8; extracting the solution with suitable solvents and acidifying the aqueous layer with mineral acid to pH below 5; separating the precipitate containing boswellic acid; washing with water till neutral to litmus; drying the resultant fraction; and optionally separating the individual boswellic acids by known methods.

Non-limiting examples of suitable polar solvents used in the extractions include alcohols (e.g., methanol, ethanol, butanol), ketones (e.g., acetone), esters (e.g., ethyl acetate), and combinations thereof. The aqueous alkali solution used for basifying may be a hydroxide compound such as sodium hydroxide, barium hydroxide or potassium hydroxide. The post-alkali treatment solvents are typically chlorinated or non-polar solvents such as dichloromethane, chloroform, hexane, petroleum ether, benzene, mixtures thereof, and the like. The mineral acid is typically one or more of hydrochloric acid, sulfuric acid, nitric acid, phosphoric acid, and the like.

The optional separation may be effected through filtration or by centrifuging. Exemplary methods for isolation of the individual acids include column chromatography, MPLC, LC, HPLC, flash chromatography, chemical techniques, and the like.

### Long Chain Polyunsaturated Fatty Acids

The nutritional compositions of the present invention comprise an omega-3 (n-3) long chain polyunsaturated fatty acid, and optionally an omega-6 (n-6) long chain polyunsaturated fatty acid such as GLA. These selected fatty acids are believed to interact with or enhance the action of the Boswellia and Phlebodium extracts as also described herein.

Long chain polyunsaturated fatty acids are those fatty acids with 18 or more carbons in an acyl chain and which also have 2 or more carbon carbon double bonds therein. Omega-3 and omega-6 fatty acids vary depending on the position of the double bond closest to the methyl end of the fatty acid. Omega-3 fatty acids have a first double bond at the third carbon and omega-6 fatty acids have a first double bond at the sixth carbon.

The selected long chain polyunsaturated fatty acids for use in the compositions of the present invention may be in any form suitable for in-vivo or in-vitro delivery or presentation of the designated fatty acid compound. Non-limiting examples of such suitable forms may include free fatty acids, fatty acid esters (e.g., esterified with an alcohol such as methanol), phospholipids, mono-, di-, or triglycerides, or combinations thereof.

Non-limiting examples of omega-3 long chain polyunsaturated fatty acids suitable for use herein include docosahexaenoic acid (DHA; 22:6n-3); eicosapentaenoic acid (EPA; 20:5n-3); stearidonic acid (18:4n-3); alpha-linolenic acid (18:3n-3); eicosatetraenoic acid; and n-3-docosapentaenoic acid. Preferred are combinations of omega-3 long chain polyunsaturated fatty acids with GLA.

The amount of such long chain polyunsaturated fatty acids in the nutritional compositions of the present invention ranges, per serving or dose, up to about 6000 mg, including from about 50 mg to about 500 mg, and also including from about 100 mg to about 250 mg, of the omega-3 fatty acid and the optional GLA. The corresponding concentration of such fatty acids most typically ranges from about 0.1% to about 10%, including from about 0.3% to about 7%, and also including from about 0.5% to about 5%, by weight of the nutritional composition.

Non-limiting examples of sources of omega-3 long chain polyunsaturated fatty acids suitable for use in the nutritional compositions include flax seed oil, canola oil, transgenic oils, and fish oil. Non-limiting examples of fish oil sources include saltwater or cold fresh water fish, non-limiting examples of which include albacore, black bass, bluefish, carp, menhaden oil, anchovy oil, pilchard oil, channel catfish, herring, lake herring, sardines, lake trout, mackerel, pompano, salmon, tuna, and white fish.

Non-limiting examples of sources of GLA or other suitable omega-6 long chain polyunsaturated fatty acid suitable for use in the nutritional compositions include primrose oil (typically 8-14% GLA), borage oil (typically 17-25% GLA), blackcurrant seed oil (14-20% GLA), transgenic GLA sources, purified GLA (typically 26-99% GLA), fungal oils (e.g., Mucor javanicus), and so forth.

### Other Macronutrients

The nutritional compositions of the present invention comprise other macronutrients including a fat source, a carbohydrate source, and a protein source, all in addition to or otherwise providing the previously described Boswellia and Phlebodium extracts and long chain polyunsaturated fatty acids.

The macronutrients in combination with the other essential or added ingredients may provide up to about 1000 kcal of energy per serving or dose, including from about 25 kcal to about 900 kcal, also including from about 75 kcal to about 700 kcal, also including from about 100 kcal to about 500 kcal, also including from about 150 kcal to about 400 kcal, and also including from about 200 kcal to about 300 kcal, per serving or dose, preferably as a single, undivided serving or dose.

Many different sources and types of proteins, lipids, and carbohydrates are known and can be used in the various nutritional products described herein, provided that the selected nutrients are safe and effective for oral administration and are compatible with the essential and other added ingredients.

Carbohydrates suitable for use in the nutritional products may be simple, complex, or variations or combinations thereof. Non-limiting examples of suitable carbohydrates include hydrolyzed or modified starch or cornstarch, maltodextrin, glucose polymers, sucrose, corn syrup, corn syrup solids, rice-derived carbohydrate, glucose, fructose, lactose, high fructose corn syrup, indigestible oligosaccharides (e.g., fructooligosaccharides), honey, sugar alcohols (e.g., maltitol, erythritol, sorbitol), and combinations thereof.

Carbohydrates suitable for use herein also include soluble dietary fiber, non-limiting examples of which include gum arabic, sodium carboxymethyl cellulose, guar gum, citrus pectin, low and high methoxy pectin, oat and barley glucans, carrageenan, psyllium and combinations thereof. Soluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof.

Proteins suitable for use in the nutritional products include hydrolyzed, partially hydrolyzed or non-hydrolyzed proteins or protein sources, and can be derived from any known or otherwise suitable source such as milk (e.g., casein, whey), animal (e.g., meat, fish), cereal (e.g., rice, corn), vegetable (e.g., soy), or combinations thereof. The proteins for use herein can also include, or be entirely or partially replaced by, free amino acids known for use in nutritional products, non-limiting examples of which include tryptophan, glutamine, tyrosine, methionine, cysteine, arginine, and combinations thereof.

Fats suitable for use in the nutritional products include coconut oil, fractionated coconut oil, soy oil, corn oil, olive oil, safflower oil, high oleic safflower oil, MCT oil (medium chain triglycerides), sunflower oil, high oleic sunflower oil, palm and palm kernel oils, palm olein, canola oil, marine oils, cottonseed oils, and combinations thereof.

The concentration or amount of carbohydrate, protein, and carbohydrate in the nutritional compositions of the present invention can vary considerably depending upon the particular product form and the various other formulations and targeted dietary needs. These macronutrients are most typically formulated within any of the caloric ranges (embodiments A, B, or C) described in the following table.

TABLE 1
Macronutrients

| | Nutritional | | |
|---|---|---|---|
| | Embodiments* | | |
| Nutrients | A | B | C |
| Carbohydrate | 1-98 | 10-75 | 30-50 |
| % total calories | | | |
| Lipid | 1-98 | 20-85 | 35-55 |
| % total calories | | | |
| Protein | 1-98 | 5-70 | 15-35 |
| % total calories | | | |

*Each numerical value is preceded by the term “about”

Optional Ingredients

The nutritional compositions of the present invention may further comprise other optional components that may modify the physical, chemical, aesthetic or processing characteristics of the products or serve as pharmaceutical or additional nutritional components when used in the targeted population. Many such optional ingredients are known or otherwise suitable for use in medical food or other nutritional products or pharmaceutical dosage forms and may also be used in the compositions herein, provided that such optional ingredients are safe for oral administration and are compatible with the essential and other ingredients in the selected product form.

Non-limiting examples of such optional ingredients include preservatives, anti-oxidants, emulsifying agents, buffers, additional pharmaceutical actives, additional nutrients as described herein, sweeteners including artificial sweeteners (e.g., saccharine, aspartame, acesulfame K, sucralose) colorants, flavors, thickening agents and stabilizers, emulsifying agents, lubricants, and so forth.

The nutritional compositions of the present invention may further comprise any of a variety of other vitamins or related nutrients, non-limiting examples of which include vitamin A, vitamin D, vitamin E, vitamin K, thiamine, riboflavin, pyridoxine, vitamin B₁₂, carotenoids (e.g., beta-carotene, zeaxanthin, lutein, lycopene), niacin, folic acid, pantothenic acid, biotin, vitamin C, choline, inositol, salts and derivatives thereof, and combinations thereof.

The nutritional compositions may further comprise any of a variety of other additional minerals, non-limiting examples of which include calcium, phosphorus, magnesium, iron, zinc, manganese, copper, sodium, potassium, molybdenum, chromium, chloride, and combinations thereof.

### Method of Use

The methods of the present invention comprise the oral administration of the nutritional compositions of the present invention, to individuals afflicted by or at risk of developing arthritis or other rheumatic diseases or conditions, to ameliorate symptoms associated with such diseases or conditions.

The method of the present invention may be applied to individuals who may or may not currently suffer from arthritis or other rheumatic or other similar inflammatory diseases or condition. Since the nutritional compositions of the present invention do not have the disadvantageous side effects of some currently available treatments for such diseases or conditions, otherwise healthy subjects may be administered the compositions with little or no change of deleterious side effects. It is believed that administration to such individuals prior to developing such diseases or conditions, especially when such individuals are at particular risk, that the onset of disease or condition can be delayed and possibly the severity and eventual progression minimized.

The methods of the present invention are used to ameliorate the symptoms of arthritis or other rheumatic diseases or condition. In this context, the term “ameliorate ” is used to define the methods of the present invention to include treating, controlling, preventing, and/or otherwise reducing the occurrence, severity or relapse of an identified symptom, condition, or disease associated with arthritis or other rheumatic disease or disorder.

The method of the present invention may therefore be used in ameliorating arthritic or other rheumatic diseases that may involve decreasing the concentration of proinflammatory cytokines in affected joint(s) in an individual by administering to the nutritional compositions described herein. Ameliorating rheumatic diseases includes, but is not limited to, delaying the onset of arthritic or other rheumatic symptoms, delaying or preventing the progression of arthritis or other rheumatic disease, and/or mitigating the severity of the symptoms of arthritis or other rheumatic disease. Thus the present invention includes methods of treating or preventing symptoms of arthritis or other rheumatic diseases, or otherwise delaying the onset of such diseases or conditions.

Examples of related methods and or uses associated with administration of the nutritional compositions include one or more of relieving or mitigating pain associated with arthritis or other rheumatic diseases, reducing the number of swollen and/or tender joints, increasing mobility/flexibility of joints, decreasing the rate at which arthritis or other rheumatic diseases progress, and relieving or mitigating the fatigue associated with such diseases or conditions.

The methods of the present invention also include a method of reducing inflammation, involving orally administering an effective amount of the nutritional composition to a subject in need of inflammation reduction, or who likely may require the need of inflammation reduction in the future (preventative maintenance). The effective amount per individual depends upon a number of factors including the severity of the symptoms and upon the responsiveness of the subject to the nutritional composition. Those of ordinary skill in the art can readily determine optimum dosages, dosing methodologies, and repetition rates.

Specific non-limiting examples of the diseases or conditions treatable by the methods of the present invention include inflammatory arthritis, rheumatoid arthritis, gout, psoriatic arthritis, reactive arthritis, viral or post-viral arthritis, spondylarthritis, rheumatism, and combinations thereof. Rheumatism includes one or more of tennis elbow, frozen shoulder, carpal tunnel syndrome, plantar fasciitis, Achilles tendonitis, and the like. Symptoms of arthritis and rheumatism include one or more of inflammation, swelling, restricted range of motion, stiffness, pain, and soreness.

The term “rheumatic disease ” as used herein, includes arthritis or other rheumatic diseases or conditions, or symptoms thereof, treatable by the oral administration of the nutritional compositions of the present invention.

The method of the present invention includes treating rheumatic diseases by administering the nutritional composition of the present invention, and further administering to the subject an effective amount of a rheumatic disease pharmacological treatment, such as an TNF-α inhibitor. Similarly, the subject invention may involve treating rheumatic diseases by administering the nutritional composition and administering to the subject a decreased amount of a rheumatic disease pharmacological treatment compared to the larger amount of the pharmacological treatment the subject would otherwise require without administration of the nutritional composition. In some instances, especially where rheumatic disease pharmacological treatments are expensive, have unwanted/undesirable side effects, or are simply difficult to obtain, administering reduced amounts (reduced dosages) of rheumatic disease pharmacological treatments is advantageous.

Non-limiting examples of rheumatic disease pharmacological treatments are described in U.S. Pat. Nos. 6,740,647; 6,207,642; and 6,171,787; which descriptions are incorporated herein by reference.

The methods of the present invention most typically involve the daily administration of the nutritional compositions of the present invention. Daily administration can be accomplished in single or divided doses or servings. Treatment is preferably maintain over prolonged periods, most typically at least about a month, including at least about 3 months, and also including periods exceeding about 6-12 months.

The methods of the present invention are most typically used in humans, but include use in other mammals such as cats, dogs, horses, cattle, and so forth.

### Manufacture

The nutritional compositions of the present invention may be prepared by any known or otherwise effective manufacturing technique for preparing the selected product form. Many such techniques are known for any given product form such as nutritional liquids, nutritional solids or bars, or pharmaceutical dosage forms (e.g., tablets, capsules, caplets, etc.,) and can easily be applied by one of ordinary skill in the art to the nutritional products described herein.

Liquid embodiments of the present invention, for example, may be prepared by first forming an oil blend containing all formulation oils, including long-chain polyunsaturated fats, and any emulsifier, fiber or fat-soluble vitamins. A carbohydrate blend and a separate protein blend are then prepared individually by mixing the carbohydrate and any minerals together, and then mixing the protein with water in a separate aqueous base. The carbohydrate and protein blends are then mixed together with the oil blend. The resulting mixture may be homogenized, heat processed, standardized with any water-soluble vitamins, flavored and the liquid terminally sterilized or aseptically filled or dried to produce a powder. The Boswellia and Phlebodium extracts can be added to any of the above-noted blends at any time during processing, although it is often desirable to select that blend in which the extracts are most soluble, and to also select that moment during processing which will result in reduced or minimal heat processing of the extracts.

Other product forms such nutritional bars may also be manufactured, for example, using cold extrusion technology as is known and commonly described in the bar manufacturing art. To prepare such compositions, typically all of the powdered components are dry blended together, which typically includes any proteins, vitamin premixes, certain carbohydrates, and so forth. The fat-soluble components, including the long chain polyunsaturated fatty acids, are then blended together and mixed with any powdered premixes. The Boswellia and Phlebodium extracts can be added at any point during formulation and processing, although it is also often desirable to select that moment during processing which will result in reduced or minimal heat processing of the extracts. Finally any liquid components are then mixed into the composition, forming a plastic like composition or dough. The resulting plastic mass can then be shaped, without further physical or chemical changes occurring, by cold forming or extrusion, wherein the plastic mass is forced at relatively low pressure through a die, which confers the desired shape. The resultant exudate is then cut off at an appropriate position to give products of the desired weight. If desired the solid product is then coated, to enhance palatability, and packaged for distribution.

The solid nutritional embodiments of the present invention may also be manufactured through a baked application or heated extrusion to produce solid product forms such as cereals, cookies, crackers, and similar other product forms. One knowledgeable in the nutrition manufacturing arts is able to select one of the many known or otherwise available manufacturing processes to produce the desired final product.

When preparing solid orally administered nutritional compositions such as capsules or tablets, the ingredients may be mixed with a pharmaceutical carrier (for example, conventional tableting ingredients such as cellulose, corn starch, lactose, sucrose, sorbitol, talc, stearic acid, magnesium stearate, dicalcium phosphate or gums) and other pharmaceutical diluents to form a solid preformulation composition containing a substantially homogenous mixture of the nutritional composition. When administered as a soft gel capsule or tablet, it is preferably swallowed with water.

Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (for example, sorbitol syrup, methyl cellulose, or hydrogenated edible fats); emulsifying agents (for example, lecithin or acacia); non-aqueous vehicles (for example, almond oil, oily esters or ethyl alcohol); preservatives (for example, methyl or propyl p-hydroxybenzoates or sorbic acid); and artificial or natural colors and/or sweeteners.

When the nutritional compositions of the present invention are in powder form, the various dry, powdered ingredients can be mixed together until a relatively homogeneous mixture is obtained. The powder is generally administered by mixing with a liquid, such as water or fruit juice, and then drinking the resulting suspension. In liquid form, the powder is mixed with an appropriate liquid carrier, such as water, and the resulting suspension is packaged in appropriate containers. In tablet form, the ingredients are mixed together and then the tablets prepared according to methods known in the art. Such methods include the wet-granulation method, the dry-granulation method, or direct compression.

The nutritional compositions of the present invention formulated or otherwise used as sole source nutritional, partial source nutritional, or as a nutritional supplement. Sole source nutritional embodiments of the present invention typically comprise fat, protein, carbohydrate, vitamins, and minerals in amounts sufficient to maintain an individual's health (such as to prevent malnutrition). Such amounts are known by those skilled in the art and can be readily calculated when preparing such formulations. For example, nutritional compositions such as Oxepa®, Ensure®, Promote®, and ProSure®, available from Ross Products Division of Abbott Laboratories, and compositions described in U.S. Pat. Nos. 6,200,624; 6,077,828; 6,066,344; 5,908,647; 5,554,589; 5,416,077; 5,223,285; 5,221,545, which are hereby incorporated by reference for their teachings of nutritional compositions (and making and using the nutritional compositions), may be combined with the Boswellia extract and/or Phlebodium extract as described herein.

As used herein, the term “Oxepa ” refers to the enteral nutrition formula commercially available from Ross Products Division, Abbott Laboratories, Columbus, Ohio, USA. This formula is a low-carbohydrate, calorically dense enteral nutrition product designed for the dietary management of critically ill patients on mechanical ventilation. It contains eicosapentaenoic acid (EPA) (from sardine oil), gamma-linolenic acid (GLA) (from borage oil), and antioxidants. OXEPA can be used as a sole source of nutrition for tube feeding.

The Oxepa enteral formula as described herein has the following caloric distribution:

| | Per 8 fl oz | Per Liter | % Calories |
|---|---|---|---|
| Calories | 355 | 1500 | — |
| Protein, g | 14.8 | 62.5 | 16.7 |
| Fat, g | 22.2 | 93.7 | 55.2 |
| Carbohydrate, g | 25.0 | 105.5 | 28.1 |
| GLA | 1.02 | 4.29 | |
| EPA | 1.08 | 4.55 | |

The Oxepa formula comprises water, sodium and calcium caseinates, sugar (sucrose), maltodextrin (corn), canola oil, medium-chain triglycerides (fractionated coconut oil), refined deodorized sardine oil, borage oil, potassium citrate, magnesium chloride, calcium phosphate tribasic, soy lecithin, sodium citrate, potassium phosphate-dibasic, ascorbic acid, natural and artificial flavor, choline chloride, taurine, d-alpha-tocopheryl acetate, L-carnitine, salt (sodium chloride), zinc sulfate, ferrous sulfate, niacinamide, carrageenan, calcium pantothenate, manganese sulfate, cupric sulfate, thiamine chloride hydrochloride, pyridoxine hydrochloride, riboflavin, beta-carotene, vitamin A palmitate, folic acid, biotin, chromium chloride, sodium molybdate, potassium iodide, sodium selenate, phylloquinone, cyanocobalamin and vitamin D3.

### EXAMPLES

The following examples illustrate the present invention. Unless otherwise indicated in the following examples and elsewhere in the specification and claims, all parts and percentages are by weight, all temperatures are in degrees Centigrade, and pressure is at or near atmospheric pressure.

Experiment 1

Tests are conducted under a model of rheumatoid arthritis in mice (collagen-induced arthritis or CIA) that shares some similarities with the human disease. The tests evaluate the effect of plant extracts and Oxepa®, on the severity of CIA and concentration of inflammatory mediators.

Seven week-old DBA/1J (H-2^{q}) male mice (n=105) are purchased from Harlan (Barcelona, Spain) and housed in plastic cages (5 animals each) with free access to food and water. After 4 days of an adapting period, the mice are paired by body weight and divided into the following groups:
1) Control: immunized mice as explained later and fed with AIN-93G diet (n=15).
2) Corticoid-treated groups: immunized mice fed with control diet and treated with intraperitoneal injections of prednisolone (5 mg/kg body weight) daily (n=15).
3) Boswellia group: immunized mice fed with the control diet supplemented with 0.5% of Boswellia serrata extract (23.47% of beta-boswellic acids) (n=15).
4) Curcuma group: immunized mice fed with the control diet supplemented with 0.5% of Curcuma longa extract (96.12% of Curcuminoids) (n=15).
5) Crataeva group: immunized mice fed with the control diet supplemented with 0.5% of Grataeva nurvala extract (80% of Lupeol) (n=15).
6) Polyphenols mixture group: immunized mice fed with the control diet supplemented with 0.9% of a mixture of green tea, grape skin, and resveratrol extracts (2:3:1) (n=15).
7) Oxepa® group: immunized mice fed with Oxepa® (n=15).

The procedure followed for the animal immunization is carried out by injecting 100 μl of bovine collagen II emulsion (type II collagen in complete Freund's adjuvant) intradermically in the base of the tail. Collagen is supplied by Chondrex (MDbiosciences, Switzerland) and the emulsion is prepared as follows: collagen is dissolved in 0.05M acetic acid (2 mg/ml) overnight at 4° C. with constant, but gentle stirring and emulsified with Freund's adjuvant (1:1). The Freund's adjuvant is added drop by drop while mixing in a high-speed homogenisator (ultraturrax) at 27,000 rpm. Only one immunization is required.

Mice are assessed daily for clinical symptoms of inflammation, and their weights are measured. The severity of clinical arthritis is graded on a nominal scale. Mice are sacrificed 10 days after the onset of arthritis.

Blood samples are taken from the retroorbital sinus under light anesthesia and the animals are immediately sacrificed by cervical dislocation. Fore and hind limbs are dissected and frozen at −80° C. Blood samples are allowed to cloth for 1 hour at room temperature and the serum is separated from blood cells by centrifugation at 6,500 g for 10 min.

The following parameters are measured in joint homogenates: antibodies against type II collagen (IgG2a); proinflammatory cytokines: IL-1β and IL-6; counter-regulatory cytokines: IL-10; and Matrix metalloproteinases: MMP-9. Antibodies against type II collagen (IgG2a) are also measured in serum.

Specific anti-CII—IgG2a in joints and serum of mice is determined by ELISA. Briefly, 96-well microtiter plates are coated overnight with CII at 4° C., washed with 0.05% Tween-PBS, blocked with 1% bovine serum albumin-PBS for 2 h at room temperature and then washed again. Samples are diluted with PBS before assay: 1:16000 for serum and 1:50 for homogenized joints. Fifty (50) μL of standards (type II collagen Ab-2, clone 2B1.5, NeoMarkers) from 0 to 500 ng/ml and samples are added to each well in duplicate, and incubated 2 h at 37° C. Wells are washed extensively, biotin-conjugated rat anti-mouse IgG2a monoclonal antibody (Pharmigen International) is added, and incubated 1 h at room temperature. After incubation, the unbound antibody is washed, avidin peroxidase is added, and incubated 30 min at room temperature. The plate is washed again, the substrate solution (ABTS) added, and incubated 12 min in dark. Finally the absorbance is measured in a microplate reader at 405 nm.

Cytokines and MMP-9 in homogenized joints are measured by ELISA using commercial kits from BIOTRAK (Amersham Pharmacia Biotech) following the supplier instructions and using joint homogenates without any further dilution.

For the statistical analysis, outliers are removed if they differed from the mean more than three standard deviations. Homogeneity of variances is analysed by Levene's test. Because the assumption of equality of variance among groups is not met, comparisons with the control group are done by unpaired t-test with Welch's correction and p values penalized by the Bonferroni's procedure and the Kruskal-Wallis's test when appropriate. The classification of arthritic index into categories is analysed by Chi-squared.

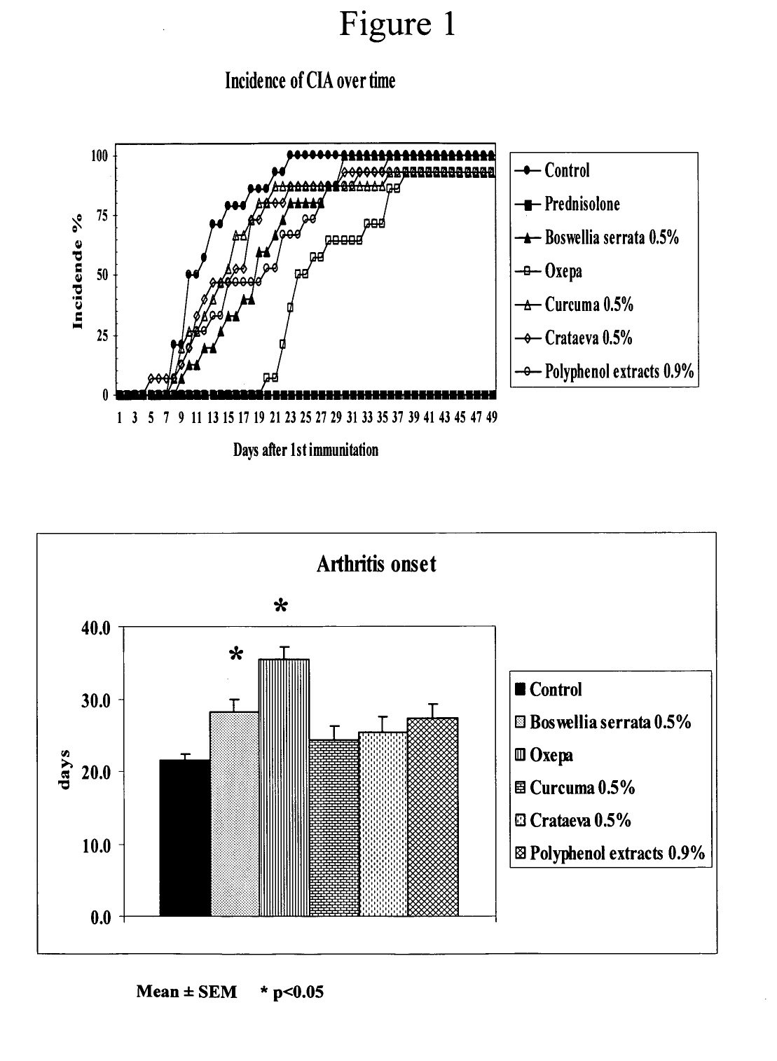

The time-course of arthritis incidence, as well as the average day of arthritis onset are shown in FIG. 1. The incidence is 100% in control, Boswellia and Polyphenol groups, and 95% in Oxepa and Curcuma groups. The mice treated with prednisolone do not develop arthritis. The arthritic onset is significantly delayed in the Boswellia and Oxepa groups, whereas only a trend is found for the Polyphenol group (p=0.111).

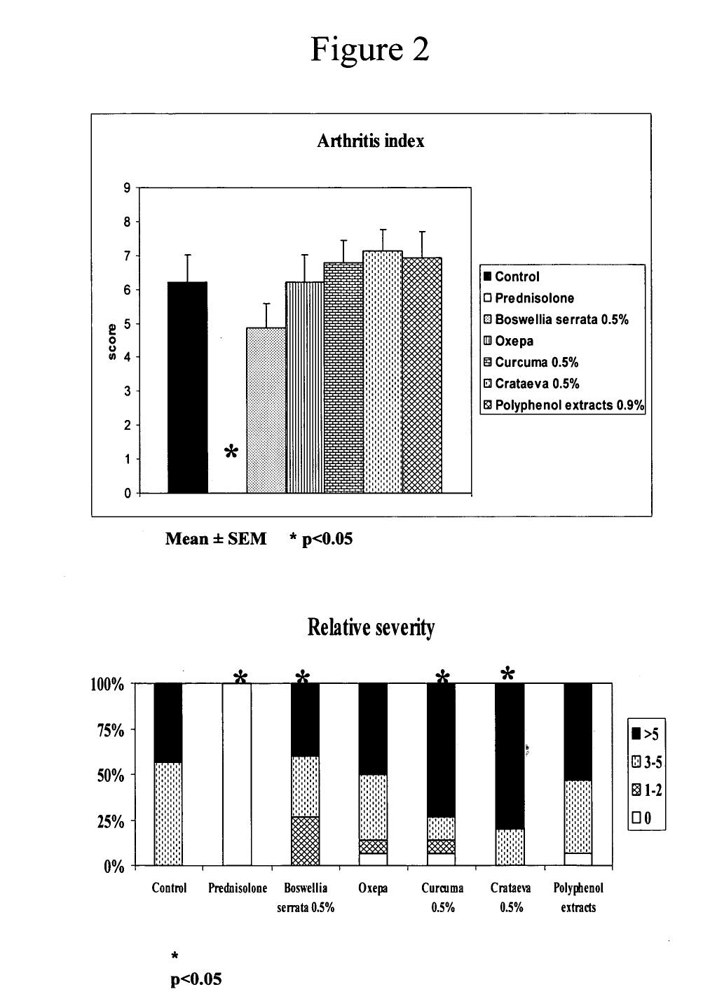

The severity of arthritis—arthritis Index and relative severity—is shown in FIG. 2. No significant differences are found between the groups receiving the diets supplemented with extracts and the control group (ANOVA p=0.265 1). However, when the arthritic index (FIG. 2) is classified into categories: 0, 1-2, 3-5 and >5, there are significant differences between the control group and the Boswellia, Curcuma and Crataeva groups. The Boswellia group has more animals with arthritis index from 1-2 than the control whereas the Curcuma and Crataeva groups have more animals with the highest of arthritis index.

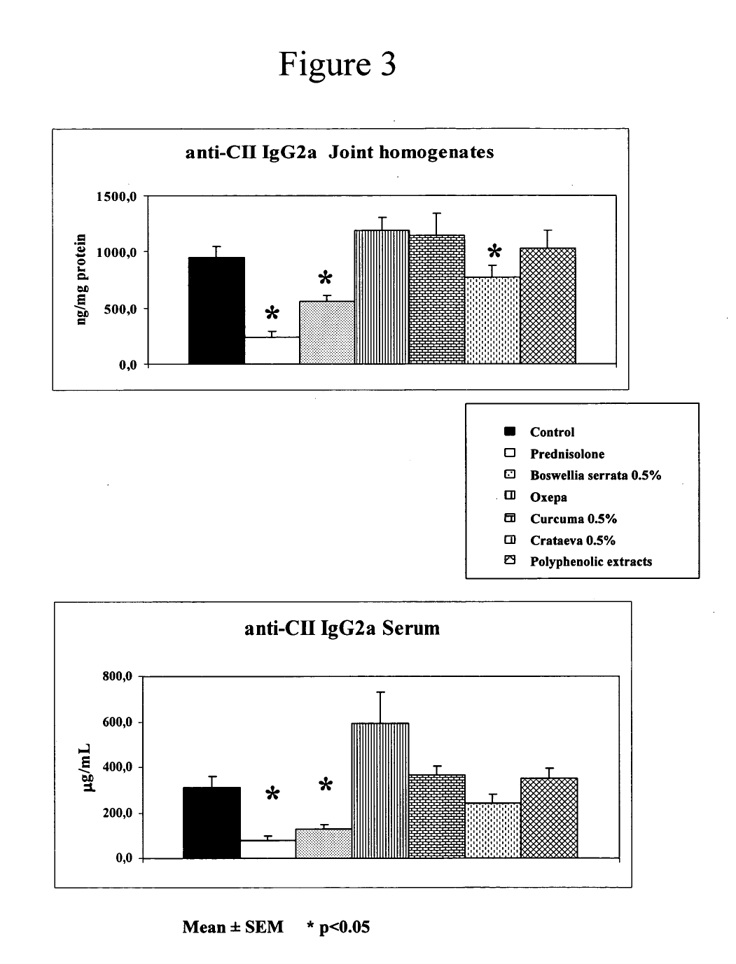

The concentration of antibodies against type II collagen (IgG2a) in serum and joint homogenates are shown in FIG. 3. A large variability is found in all groups, except in the Prednisolone and Boswellia groups. The IgG2a levels in both serum and joint homogenates are significantly lower in prednisolone and Boswellia groups with respect to the control group. In addition, the content of antibodies against type II collagen (IgG2a ) in joint homogenates is lower in the Crataeva group than in the control group.

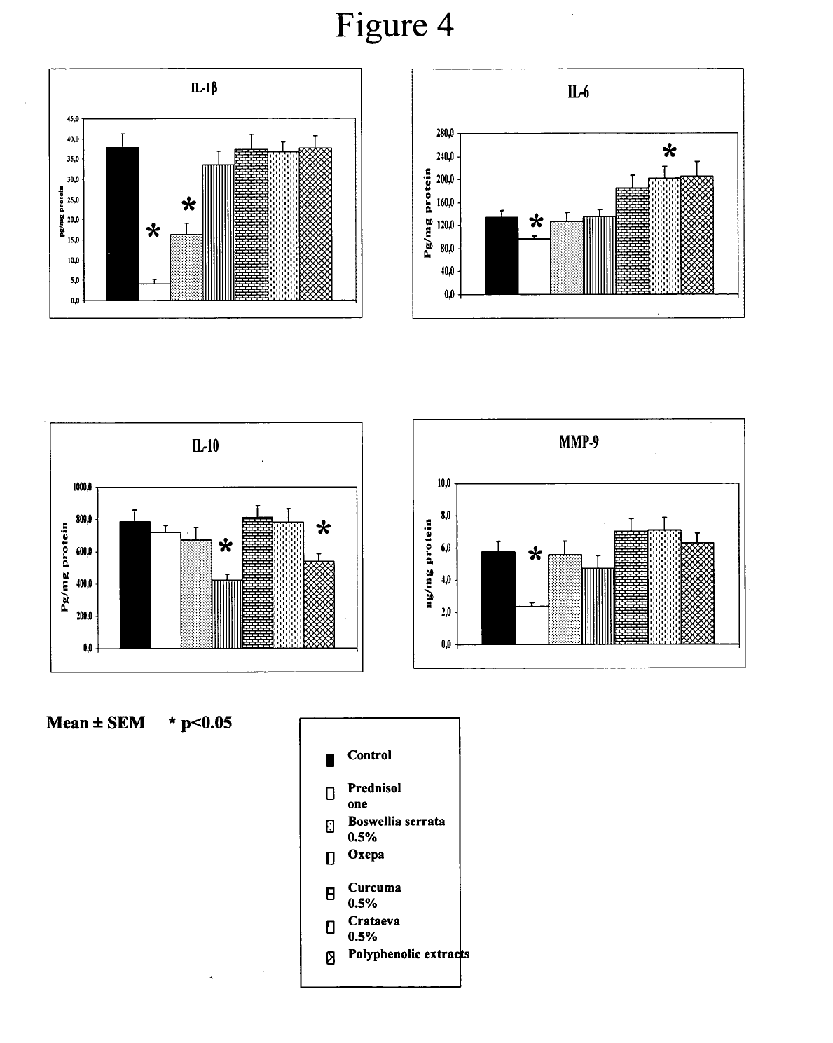

The concentrations of IL-1β, IL-10, and MMP-9 in joint homogenates of study groups are shown in FIG. 4. The content of inflammatory mediators (IL-1β, IL-6), and of MMP-9 is lower in the group of mice treated with corticoids than in controls. The content of IL-1β is lower in Boswellia group than in the control group. The groups fed with Crataeva and Polyphenol supplemented diets tended to have higher concentration of IL-6 in joint homogenates than controls. The content of IL-10 is lower in Polyphenol extract and Oxepa® groups than in the control group.

Conclusions

no adverse effects are observed in mice fed with diets supplemented with 0.5% Boswellia serrata extract, 0.5% Grataeva nurvala extract, 0.5% Curcuma longa extract, or 0.9% polyphenol mixture of green tea, grape skin and resveratrol (2:3:1). No adverse effects are observed in mice fed with Oxepa®.

the supplementation of diet with Crataeva nurvala and Curcuma longa extract do not show any effect on collagen-induced arthritis in mice.

The supplementation of the diet with 0.5% of Boswellia serrata has beneficial effects on the development of collagen-induced arthritis in mice, showing a pattern of less severe disease than control animals. The addition of this extract to the diet: 1) delays significantly the day of arthritis onset, 2) decreases the content of IgG2a both in joint homogenates and serum, 3) decreases the content of the pro-inflammatory cytokine IL-1β in joint homogenates, and 4) reduces number of animals with high score of arthritic index.

Feeding with Oxepa® does not reduce clinical symptoms of inflammation or inflammatory mediators. However, it delays the day of arthritis onset.

Experiment 2

A second test is performed to test a Phlebodium decumanum extract in CIA mice. Mice are treated in accordance with the Experiment 1 model. The following two groups are studied:
1) Control: immunized mice fed with AIN-93G diet (n=15).
2) Phlebodium group: immunized mice fed with the control diet supplemented with 0.5% of Phlebodium decumanum extract (n=15).

The clinical data are obtained from all the animals whereas the biochemical data are from only 5 animals per group. Clinical parameters include: incidence, day of arthritic onset, arthritic index and relative severity. Biochemical parameters in serum include: antibodies against type II collagen (IgG2a). Biochemical parameters in joints include: antibodies to Type II collagen (IgG2a), IL-1β, IL-6, and IL-10.

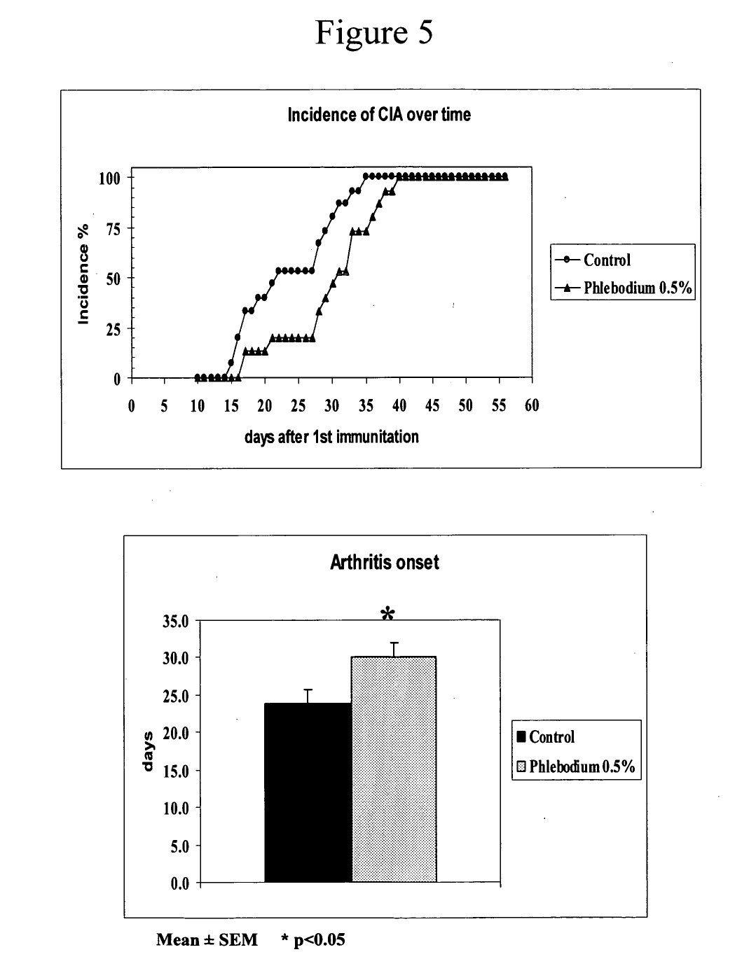

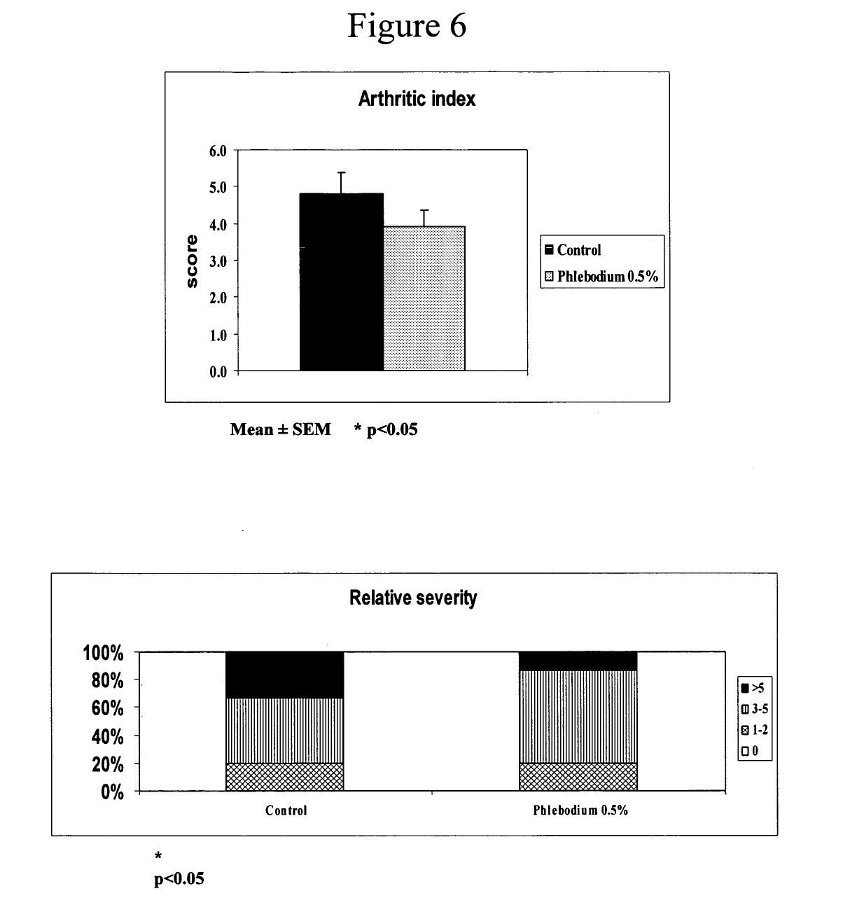

The time-course of arthritis incidence, as well as the average day of arthritis onset are shown in FIG. 5. The incidence is the same in the control and the Phlebodium group (100%) although the arthritic onset is significantly delayed in the latter. There are no significant differences on the total severity (arthritic index) or the relative severity of arthritis. However, the average arthritis index is lower in the Phlebodium group due to the lower percentage of animals with the higher score (FIG. 6).

Table 2 shows concentrations of antibodies to type II collagen (IgG2a) in serum and joint homogenates. It also shows concentrations of inflammation mediators (IL-1β, IL-6 and IL-10) in joint homogenates. The levels of antibodies to type II collagen (IgG2a), IL-1β, IL-6 are within range of previous experiments whereas those of IL-10 are lower than those previously reported. No significant differences are found between the control and the Phlebodium group (i.e., among type II collagen-immunized mice fed, those fed a control diet or the same diet supplemented with Phlebodium extract).

TABLE 2
Experiment 2 Data

| Component | Control¹ | Phlebodium¹ |
|---|---|---|
| IgG2a (serum, μg g/ml) | 393.1 ± 260.3 | 444.4 ± 181.5 |
| IgG2a (joints, ng/mg protein) | 1082.5 ± 1376.0 | 1234.5 ± 984.2 |
| IL-1β (joints, pg/mg protein) | 44.1 ± 14.7 | 57.2 ± 30.6 |
| IL-6 (joints, pg/mg protein) | 226.5 ± 95.4 | 304.8 ± 100.4 |
| IL-10 (joints, pg/mg protein) | 337.1 ± 83.32 | 347.8 ± 70.68 |

¹Data expressed as mean ± SD.

In conclusion, no adverse effects are observed in mice fed with a diet supplemented with 0.5% of Phlebodium decumanum extract. The addition of a Phlebodium decumanum extract to the diet delayed significantly the day of arthritis onset, without affecting the severity of the disease. This effect is not explained by any alteration on the levels of the inflammatory parameters measured.

Experiment 3

Experiment 1 is repeated and the test animals evaluated for various histological changes associated with each control or treatment group. The purpose of this experiment is to show the effect of Boswellia serrata and Phlebodium decumanum extracts and Oxepa® on the histological structure of cartilage and bone in the same experimental model as described above.

After again completing the above-described model, the mice are sacrificed 10 days after the arthritis onset by cervical dislocation under light anesthesia. Fore and hind limbs are dissected and the skin removed. Each is then immersed in 4% buffered formaldehyde for 2-3 days. Afterwards, each is treated with decalcificant solution for 2 hours and treated again for 60-90 minutes more. The samples are dehydrated with alcohol solutions of increasing concentrations, embedded in paraffin, cut into 4 μm slices and stain with hematoxilin and eosin (H&E).

The following joints are thereafter studied: elbow, radio-cubito-carpo (wrist), carpo-metacarpo and metacarpo-phalange in fore limbs, and knee, tibio-tarso (ankle), tarso-metatarso, metatarso-phalange in hind limbs. Typical alterations of arthritic joints are considered, such as synovial inflammation, pannus formation, cartilage damage and bone destruction, and are scored on a nominal scale from 0 to 3. The scale is defined for each parameter as follows:

Synovial inflammation: 0=Absence: normal synovial-periarticular tissue; 1=mild: infiltration of inflammatory cells in the periarticular tissue; 2=moderated: cell infiltration and moderated edema; 3=severe: marked cell infiltration and edema.

Pannus formation: None (0), mild (1), moderate (2) or severe (3) proliferation of synovial tissue at the synovium-cartilage junction.

Cartilage damage: 0=Absence: normal cartilage; 1=mild: damage of Zone I (external) with slight loss of chondrocytes and/or collagen disruption; 2=moderated: damage of Zone II (internal) with moderate loss of chondrocytes and/or collagen disruption: 3=severe: severe damage of all Zones with multifocal losses of chondrocytes and/or collagen disruption.

Subchondral bone destruction: 0=Absence: normal bone; 1=mild: some areas of cortical degradation, few osteoclasts; 2=moderated: clear bone degradation with moderate medular damage, more osteoclasts; 3=severe: intense cortical and medular bone destruction, numerous osteoclasts.

The scores of all joints are summed to obtain a general score for each histological parameter and animal. The histopathologist is not aware of the study groups during the evaluation of the samples.

Statistical Analysis

Homogeneity of variances are tested by the Bartelett's test. If homogeneity of variances is met, data are analysed by one-way ANOVA. If variances are not homogeneous, non-parametric procedures are used. The analysis is done by the Graph Pad Prism Software version 4.

Results

Two mice died during the immunization procedure, one from the corticoid group and one from the Oxepa® group. Consequently, 28 animals are evaluated. The mice treated with prednisolone do not develop arthritis, while the incidence of arthritis over time in the Control, Polyphenol, and Phlebodium groups are 100%. One mouse in the Boswellia group and one in the Oxepa group does not develop the disease.

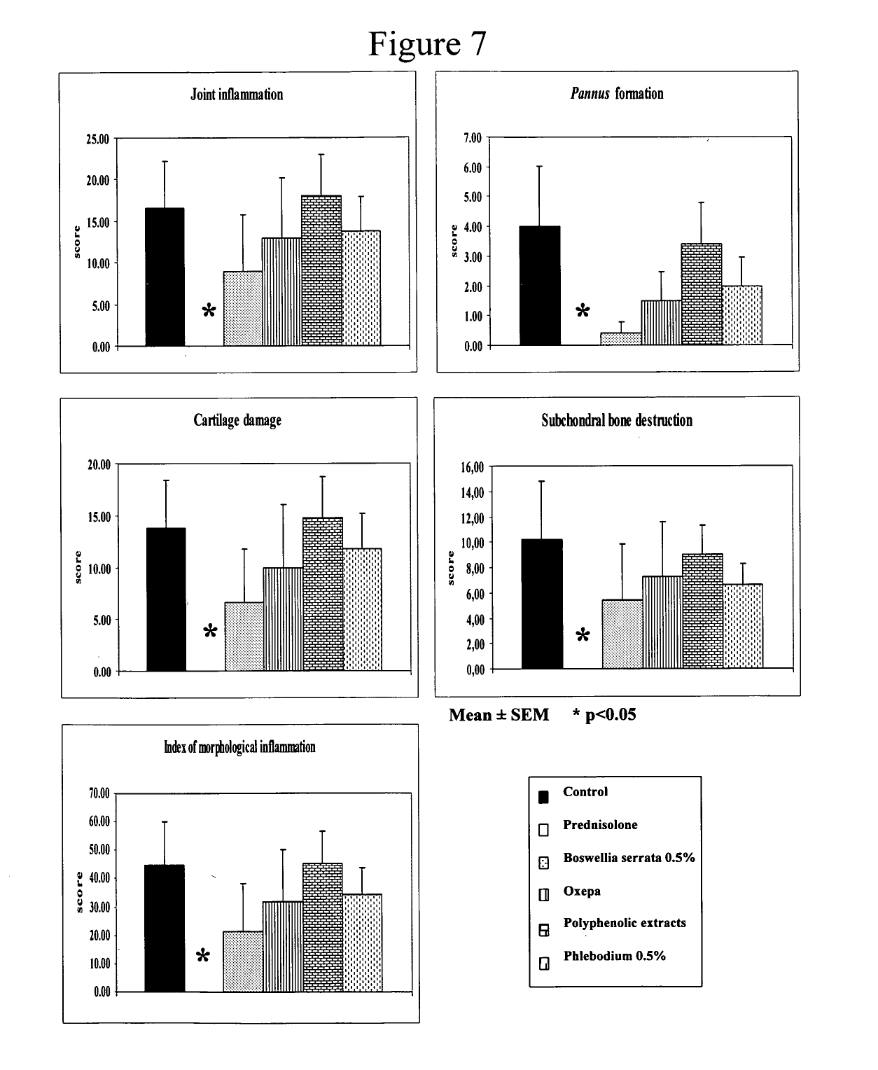

The data of arthritis index, arthritis onset, and the total scores for inflammation, pannus formation, cartilage damage and bone destruction are shown in Table 3. The average values for each score are shown in FIG. 7, as well as an index of morphological inflammation, which was obtained by summing the scores of the 4 histological parameters.

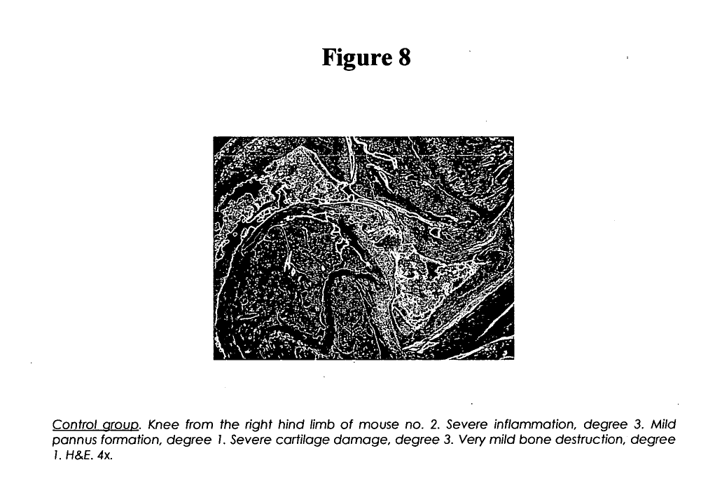

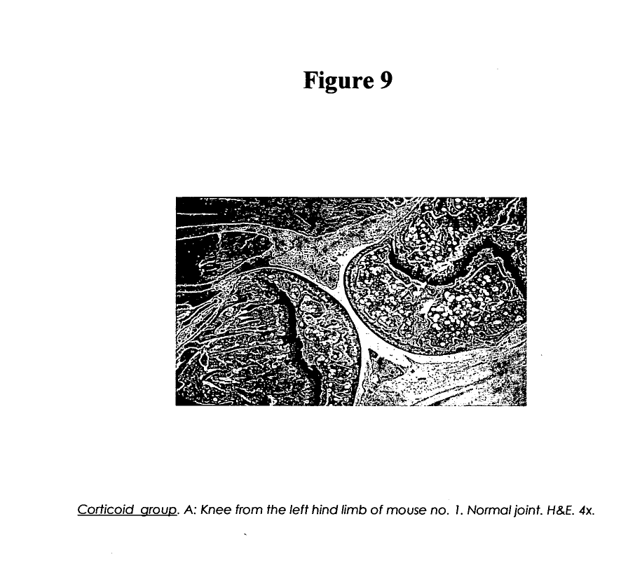

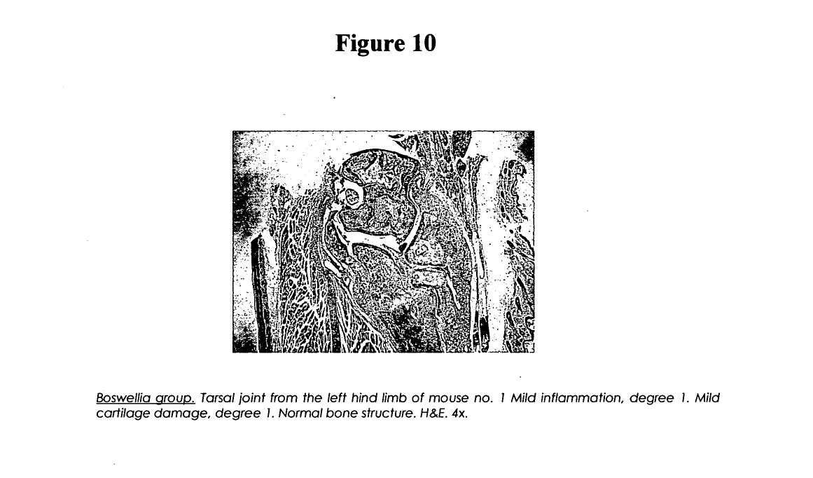

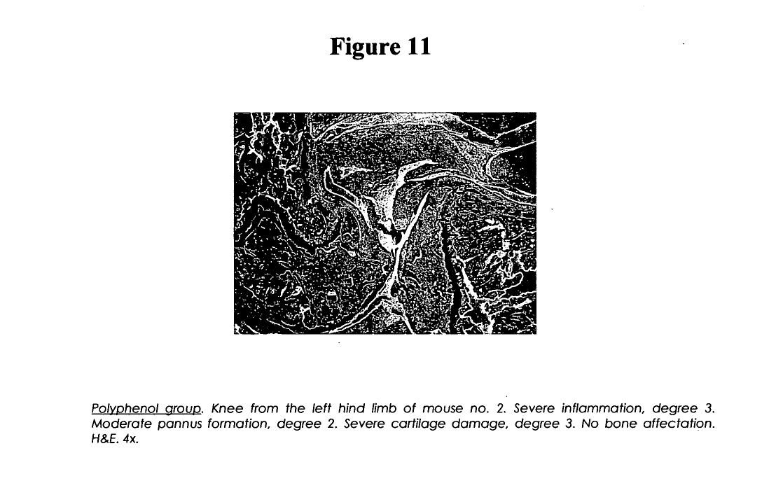

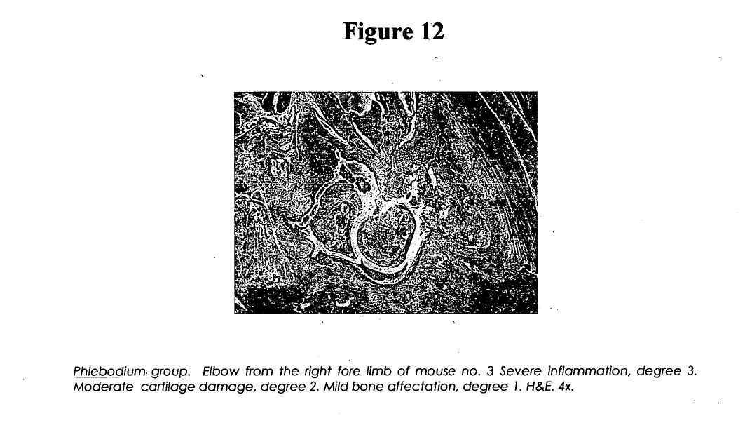

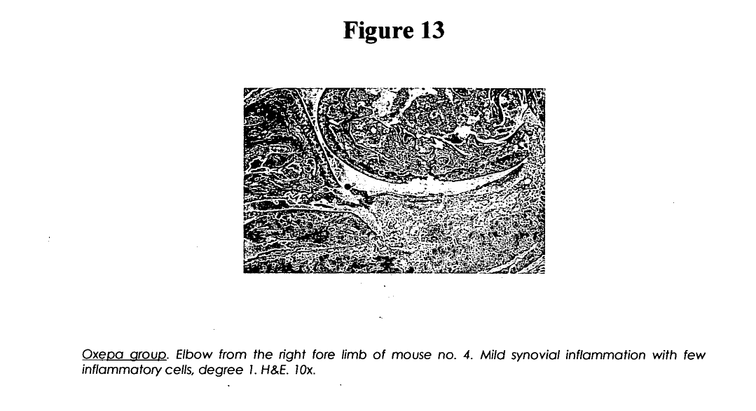

As show below, Table 3 set forth data directed to arthritis index, day of arthritis onset and histological scores in type II collagen-immunized mice fed with control diet, the same diet supplemented with Boswellia serrata 0.5%, polyphenol mixture of green tea, grape skin, and resveratrol (2:3:1) 0.9%, Phlebodium decumanum 0.5% and Oxepa. The corticoid group are immunized mice fed with the control diet and treated intraperitoneally with prednisolone daily. Histological slides from the study animals are illustrated in FIGS. 8-13.

TABLE 3
Experiment 3 Data

| | | | Arthritis | | | | |
|---|---|---|---|---|---|---|---|
| | | Arthritis | onset | Inflammation | Pannus | Cartilage | |
| Group | Mouse No. | index | (days) | score | score | score | Bone score |
| Control | 1 | 8 | 28 | 30 | 2 | 25 | 25 |
| | 2 | 3 | 17 | 6 | 3 | 4 | 1 |
| | 3 | 7 | 20 | 18 | 2 | 17 | 13 |
| | 4 | 1 | 33 | 2 | 1 | 2 | 0 |
| | 5 | 7 | 22 | 27 | 12 | 21 | 12 |
| Corticoid | 1 | 0 | — | 0 | 0 | 0 | 0 |
| | 2* | — | — | — | — | — | — |
| | 3 | 0 | — | 0 | 0 | 0 | 0 |
| | 4 | 0 | — | 0 | 0 | 0 | 0 |
| | 5 | 0 | — | 0 | 0 | 0 | 0 |
| Boswellia | 1 | 2 | 37 | 4 | 0 | 3 | 1 |
| | 2 | 2 | 17 | 0 | 0 | 0 | 0 |
| | 3 | 0 | | 0 | 0 | 0 | 0 |
| | 4 | 12 | 30 | 36 | 2 | 27 | 23 |
| | 5 | 4 | 18 | 5 | 0 | 3 | 3 |
| Polyphenols | 1 | 5 | 31 | 27 | 1 | 21 | 12 |
| | 2 | 5 | 34 | 15 | 8 | 14 | 12 |
| | 3 | 7 | 18 | 25 | 4 | 17 | 9 |
| | 4 | 8 | 17 | 23 | 4 | 22 | 12 |
| | 5 | 1 | 27 | 0 | 0 | 0 | 0 |
| Phlebodium | 1 | 3 | 17 | 12 | 2 | 10 | 8 |
| | 2 | 4 | 33 | 16 | 5 | 17 | 10 |
| | 3 | 7 | 40 | 26 | 3 | 19 | 8 |
| | 4 | 1 | 21 | 0 | 0 | 0 | 0 |
| | 5 | 4 | 31 | 15 | 0 | 13 | 7 |
| Oxepa | 1* | — | — | — | — | — | — |
| | 2 | 0 | | 2 | 0 | 0 | 0 |
| | 3 | 1 | 19 | 0 | 0 | 0 | 0 |
| | 4 | 5 | 45 | 21 | 2 | 16 | 12 |
| | 5 | 6 | 13 | 29 | 4 | 24 | 17 |

*mice that die during the immunization procedure.

Although the day of arthritis onset is similar among groups, the development of the illness is somewhat delayed, especially in the Oxepa® group, resembling the results of the previous experiments described herein. No significant differences are found due to the high variability and the low number of individuals in each group. In the Boswellia group, animal number 4 shows a different behavior to the other animals within this group, being the source of higher variability in this group and not allowing the detection of significant differences with respect to the control group. However, on average, lower scores are found for the Boswellia group, followed by the Oxepa® group and the Phlebodium group.

Boswellia and Oxepa are more effective in inhibiting the development of pannus. In fact, the addition of Boswellia extract to the diet produces nearly a complete inhibition of pannus formation in this group even for the animal having the higher arthritic index (arthritic index 12, pannus formation 2).

Conclusions

High intra-group variability and small sample size does not allow for detection of significant group differences in Experiment 3. However, taking into consideration each histological parameter individually or a sum of scores for each animal, the order of the groups according to the degree of histological damage is Boswellia<Oxepa®<Phlebodium<Control<Polyphenol. These results are in agreement with our previous report showing a beneficial effect of Oxepa and a powder rodent diet supplemented with Boswellia serrata or Phlebodium decumanum at 0.5% on the development of rheumatoid arthritis in collagen-induced arthritis in mice.

While the invention has been explained in relation to certain embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A nutritional composition for treating rheumatic diseases, comprising:
(A) a fat source comprising at least one of:
(i) at least one omega-3 long chain polyunsaturated fatty acid and
(ii) at least one omega-6 long chain polyunsaturated fatty acid;
(B) a carbohydrate source;
(C) a protein source; and
(D) from about 0.1% to about 5% by weight of at least one of a Boswellia extract and a Phlebodium extract.

2. The nutritional composition of claim 1 comprising from about 0.1% to about 5% by weight of the Boswellia extract and the Phlebodium extract.

3. The nutritional composition of claim 1, wherein the Phlebodium extract comprises a 3Phlebodium decumanum extract.

4. The nutritional composition of claim 1, wherein the Boswellia extract comprises a Boswellia serrata extract.

5. The nutritional composition of claim 1 comprising from about 0.2% to about 3% by weight of at least one of a Boswellia extract and a Phlebodium extract.

6. The nutritional composition of claim 1 in a form of one of a bar and a powder.

7. The nutritional composition of claim 1 in a liquid form.

8. The nutritional composition of claim 1, wherein the fat source comprises at least one of eicosapentaenoic acid, stearidonic acid, docosahexanoic acid, and alpha-linolenic acid.

9. The nutritional composition of claim 1, wherein the fat source comprises gamma-linolenic acid.

10. The nutritional composition of claim 1 comprising the Boswellia extract, the Boswellia extract comprises at least 25% by weight of one or more boswellic acids.

11. The nutritional composition of claim 1, wherein the at least one of a Boswellia extract and a Phlebodium extract comprises at least one of a woody plant of the family Burseraceae and a fern plant of the Family Polypodiaceae.

12. A method of treating rheumatic diseases, comprising administering to a subject an effective amount of a composition comprising a fat source comprising at least one omega-3 long chain polyunsaturated fatty acid or least one omega-6 long chain polyunsaturated fatty acid, a carbohydrate source, a protein source, and from about 0. 1% to about 5% by weight of at least one of a Boswellia extract and a Phlebodium extract.

13. The method of claim 12, wherein treating rheumatic diseases involves decreasing a concentration of proinflammatory cytokines in joint homogenates in the subject.

14. The method of claim 12, wherein the rheumatic diseases comprise at least one selected from the group consisting of inflammatory arthritis, rheumatoid arthritis, gout, psoriatic arthritis, reactive arthritis, viral or post-viral arthritis, spondylarthritis, osteoarthritis, and rheumatism.

15. The method of claim 12 further comprising administering to the subject an effective amount of an TNF-α inhibitor.

16. The method of claim 12, wherein treating rheumatic diseases involves delaying an onset of arthritis symptoms.

17. The method of claim 12, wherein the composition comprises from about 0.1% to about 5% by weight of the Boswellia extract and the Phlebodium extract.

18. The method of claim 12, wherein the Phlebodium extract comprises a Phlebodium decumanum extract.

19. The method of claim 12, wherein the Boswellia extract comprises a Boswellia serrata extract.

20. The method of claim 12, wherein the composition is administered to the subject orally.
